(19) United States
(12) Patent Application Publication
(10) Pub. No.: US 2022/0008512 A1
(43) Pub. Date: Jan. 13, 2022

(54) ANTI-CANCER MONOTHERAPY USING SA-4-1BBL

(71) Applicants: UNIVERSITY OF LOUISVILLE RESEARCH FOUNDATION, INC., Louisville, KY (US); FASCURE THERAPEUTICS, LLC, Louisville, KY (US)

(72) Inventors: Haval SHIRWAN, Columbia, MO (US); Esma S. YOLCU, Columbia, MO (US); Rajesh SHARM, San Diego, CA (US)

(73) Assignees: UNIVERSITY OF LOUISVILLE RESEARCH FOUNDATION, INC., Louisville, KY (US); FASCURE THERAPEUTICS, LLC, Louisville, KY (US)

(21) Appl. No.: 17/294,157

(22) PCT Filed: Nov. 15, 2019
(86) PCT No.: PCT/US2019/061594

§ 371 Date: May 14, 2021

Related U.S. Application Data
(60) Provisional application No. 62/767,901, filed on Nov. 15, 2018.

(51) Int. Cl. A61K 38/19; A61K 9/00; A61P 35/00

(52) U.S. Cl. CPC A61K 38/191; A61P 35/00; A61K 9/0019

(57) ABSTRACT

Described herein are methods for treating and preventing cancer. In particular, described herein are methods using SA-4-1BBL as a monotherapy agent to treat, prevent or reduce the risk of cancer, treat, prevent, or reduce the risk of tumorigenesis, and treat, prevent, or reduce the risk of post-surgical tumor recurrence. SA-4-1BBL for use as a monotherapy in such methods also is provided.

### CROSS-REFERENCE TO RELATED APPLICATIONS

This application claims the priority benefits of U.S. provisional application 62/767,901, filed Nov. 15, 2018, the entire contents of which are incorporated herein by reference in their entirety.

### STATEMENT REGARDING FEDERALLY SPONSORED RESEARCH

This invention was made with government support under Grant No. R41CA199956 awarded by the National Institutes of Health. The government has certain rights in the invention.

### FIELD

Described herein are methods for treating and preventing cancer.

### BACKGROUND

4-1BB (CD137; TNFRSF9) is a potent T-cell costimulatory receptor that belongs to the tumor necrosis factor receptor superfamily. 4-1BB is primarily expressed on the surface of activated lymphoid cells, including T cells, B cells, and NK cells. Signaling through 4-1BB has been shown to result in the survival, expansion, and differentiation of T cells, particularly CD8⁺ T cells, into effectors and the establishment of long-term memory. Given the demonstrated role of CD8⁺ T cells as important effectors of cancer immunotherapy, the 4-1BB pathway has been the subject of intense basic and translational research in the immuno-oncology field. Agonistic antibodies (Abs) to 4-1BB alone or in combination with other anti-cancer agents have shown therapeutic efficacy in various preclinical models, which led to efforts of translating these findings into the clinic. Presently, there are ongoing clinical trials to evaluate the efficacy of agonistic 4-1BB antibodies alone or in combination with other immunotherapy and chemotherapy modalities. However, the use of agonistic antibodies to 4-1BB was reported to cause significant hepatic toxicity and other complications in preclinical as well as clinical studies. Importantly, treatment with agonistic antibodies was shown to have deleterious effects on various immune cells, including CD4⁺ T cells, humoral immune responses, and NK cells. It is presently unknown if these adverse effects are bona fide physiological characteristics of 4-1BB receptor signaling or complications associated with the use of agonistic antibodies.

There is only one known natural 4-1BB ligand (4-1BBL) expressed as a type II transmembrane protein primarily on antigen presenting cells, such as dendritic cells (DCs), macrophages, and B cells. The membranous form of 4-1BBL exists as a trimer, and upon engagement with its receptor on T cells it delivers a robust costimulatory signal. In marked contrast, the trimeric soluble form of 4-1BBL lacks costimulatory functions and requires cross-linking to either solid surfaces or by other means to acquire costimulatory function.

We previously generated a recombinant chimeric protein, SA-4-1BBL, containing the extracellular domains of murine or human 4-1BBL fused to a modified form of streptavidin core. SA-4-1BBL forms tetramers and oligomers with robust T cell costimulatory activity in soluble form. We showed that SA-4-1BBL blocked the conversion of T conventional cells into CD4⁺CD25⁺Foxp3⁺ T regulatory cells (Tregs) that was dictated by the production of IFN-γ in T conventional cells. SA-4-1BBL also overcame Treg suppression by stimulating the production of IL-2 in T effector cells (Teffs). Importantly, treatment with SA-4-1BBL did not result in various immune system anomalies in mice, such as systemic cytokine storm, splenomegaly, lymphadenopathy, and hepatitis, otherwise reported for 4-1BB antibodies.

We previously showed that, as an adjuvant component of tumor-associated antigen-based subunit vaccines, SA-4-1BBL generated robust T effector responses with therapeutic efficacy in various preclinical tumor models.

However, there remains a need for effective anti-cancer and anti-tumor therapies.

### SUMMARY

Provided herein is a monotherapy method of preventing or treating a cancer, comprising administering to a subject in need thereof an effective amount of SA-4-1BBL, wherein the monotherapy method does not include administering to the subject an antigen associated with the cancer.

Also provided herein is a monotherapy method of reducing the risk of tumorigeneses, comprising administering to a subject in need thereof, an effective amount of SA-4-1BBL, wherein the monotherapy method does not include administering to the subject an antigen associated with the tumor.

Also provided herein is a monotherapy method of reducing the risk of tumor recurrence, such as after cancer or tumor treatment, comprising administering to a subject in need thereof, an effective amount of SA-4-1BBL, wherein the monotherapy method does not include administering to the subject an antigen associated with the tumor.

Also provided herein is SA-4-1BBL for a monotherapy method for preventing or treating a cancer, or for reducing the risk of tumorigeneses, or for reducing the risk of tumor recurrence, such as after cancer or tumor treatment, wherein the monotherapy method does not include administering to the subject an antigen associated with the cancer.

In accordance with any of the methods or uses described herein, the subject may be at risk of developing cancer due to lifestyle risk factors such as obesity, smoking, and/or alcohol use, or exposure to environmental risk factors, such as asbestos, UV radiation, and/or ionizing radiation.

In accordance with any of the methods or uses described herein, the subject may be suffering from chronic liver disease, have a hereditary mutation in a p53 gene, have a hereditary mutation in a breast cancer gene, have a DNA repair deficiency, and/or have preneoplastic or early neoplastic lesions.

In accordance with any of the methods or uses described herein, the subject may have lifestyle risk factors, environmental risk factors, a history of hereditary BRCA1 and/or BRCA2 mutations, Lynch Syndrome, Cowden Syndrome, and/or be infected with HPV.

In accordance with any of the methods or uses described herein, the subject may suffer from, be at risk of developing, or have been treated for melanoma, lymphoma, lung cancer, or breast cancer.

In accordance with any of the methods or uses described herein, the SA-4-1-BBL may be administered by intravenous, subcutaneous, or intraperitoneal injection.

In accordance with any of the methods or uses described herein, the SA-4-1-BBL may be administered twice, two weeks apart, optionally followed by a rest period of 2-6 months, after which the SA-4-1-BBL is administered twice, two weeks apart.

In accordance with any of the methods or uses described herein, the SA-4-1-BBL may comprise the amino acid sequence of the extracellular domain of human 4-1-BBL, including the amino acid sequence set forth in FIG. 8B. The SA-4-1-BBL may have the amino acid sequence set forth in FIG. 8A or FIG. 8B.

Also provided herein is an SA-4-1-BBL conjugate having the amino acid sequence set forth in FIG. 8A or FIG. 8B, a composition comprising same and a carrier, a pharmaceutical composition comprising same and a pharmaceutically acceptable carrier, such as a pharmaceutically acceptable carrier suitable for administration by intravenous, subcutaneous, or intraperitoneal injection.

### BRIEF DESCRIPTION OF THE DRAWINGS

FIG. 1A-D show that pretreatment with SA-4-1BBL in the absence of any TAA antigen confers protection against tumor. FIG. 1A shows the experimental design and timeline. FIG. 1B shows anti-SA antibody titers of C57BL/6 mice pretreated twice with 25 μg SA-4-1BBL (days 0 and 14), followed by TC-1 tumor challenge (1×10⁵) on day 28. A group of mice was also vaccinated subcutaneously (s.c.) with 50 μg E7-P1 peptide+25 μg SA-4-1BBL 8 days post-tumor challenge. Serum was collected on days 21, 34, and 60 post-treatment with the initial dose of SA-4-1BBL and anti-SA antibody titers were assessed with ELISA. FIG. 1C shows that anti-SA antibodies do not block the costimulatory function of SA-4-1BBL in vitro. SA-4-1BBL protein was incubated at different doses (μg/ml) with naïve or immune serum with high titers (Log₁₀=2.8) of anti-SA antibodies for 1 hour at room temperature. The costimulatory activity of SA-4-1BBL pre-incubated with serum was then assessed using an in vitro CD3-based T cell proliferation assay. SA-4-1BBL and SA-4-1BBL preincubated with naïve serum were used at similar doses as controls. FIG. 1D shows Kaplan-Meier survival curves of mice subjected to different treatments. Mice pretreated with SA-4-1BBL alone and those pretreated with SA-4-1BBL followed by immunization with SA-4-1BBL-adjuvanted E7-P1 subunit vaccine according to the experimental scheme in FIG. 1A were monitored for tumor growth. Mice were euthanized when average tumor size reached ≥12 mm in diameter or on day 60 post-tumor challenge as experimental end-point. Data was analyzed using Kaplan-Meier Survival curve and log-rank statistical method. A P value ≤0.05 was considered significant.

FIG. 2A-B show time-course dynamics of the SA-4-1BBL generated anti-tumor responses. FIG. 2A shows that C57BL/6 mice were pretreated once with 25 μg SA-4-1BBL followed by TC-1 challenge (1×10⁵ cells) at the indicated time points. FIG. 2B shows that pretreatment with SA-4-1BBL twice, two weeks apart, followed by TC-1 challenge at the indicated time points. Mice were monitored for tumor growth and euthanized when average tumor size reached ≥12 mm in diameter or at the 60-day experimental endpoint. Data was analyzed using Kaplan-Meier Survival curve and log-rank statistical method. A P value ≤0.05 was considered significant.

FIG. 3A-D show that prevention conferred by SA-4-1BBL is dose-dependent and effective against different tumor types. FIG. 3A shows the experimental design and timeline. FIG. 3B shows that pretreatment with SA-4-1BBL protects mice against different tumor types. C57BL/6 mice were pretreated with 25 μg SA-4-1BBL twice, 2 weeks apart, and challenged 2 weeks later with LLC or TC-1 tumor cells. Tumor measurements were recorded twice weekly using calipers and mice were euthanized when tumor size reached ≥12 mm in diameter. FIG. 3C shows that pretreatment with SA-4-1BBL protected mice against tumor challenge in a dose-dependent manner. C57BL/6 mice were pretreated with the indicated doses of SA-4-1BBL twice, two weeks apart. Mice were challenged with the 3LL-huMUC1 tumor cells one week post-SA-4-1BBL treatment and then monitored for tumor growth. FIG. 3D shows that the prophylactic efficacy of SA-4-1BBL results in moderate immune memory response against tumor. Mice were pretreated with SA-4-1BBL (25 μg/injection, twice, 2 weeks apart) and two weeks later challenged s.c. with 1×10⁵TC-1 tumor cells. A second group of animals treated the same were vaccinated s.c. with SA-4-1BBL-adjuvanted E7 P1 peptide (50 μg peptide+25 μg SA-4-1BBL) 8 days after tumor challenge. Mice in both groups were monitored for 60 days, and those without tumor were re-challenged with a second dose of TC-1 tumor cells to assess immune memory. Animals were monitored for additional 80 days. Data was analyzed using Kaplan-Meier Survival curve and log-rank statistical method. A P value ≤0.05 was considered significant.

FIG. 4A-D show that SA-4-1BBL is a bona fide novel anti-tumor immunomodulator, and humoral immunity is dispensable for its prophylactic effect against tumors. FIG. 4A shows that pretreatment with 3H3 agonist 4-1BB antibody does not result in protection against tumor. C57BL/6 mice were pretreated s.c. twice, two weeks apart with streptavidin alone (12.5 μg), 3H3 alone (100 μg), streptavidin+3H3, or SA-4-1BBL (25 μg), followed by TC-1 (1×10⁵) challenge s.c. 2 weeks after the last treatment. Animals were monitored for tumor growth and euthanized when tumor size reached ≥12 mm or at the 60-day experimental endpoint. FIG. 4B shows anti-streptavidin antibody titers in serum from mice in FIG. 4A pretreated with streptavidin alone, or streptavidin+3H3 antibody collected on day 21 or experimental end-point. FIG. 4C shows that passive transfer of immune serum with high titers against streptavidin into naïve mice does not protect against subsequent tumor challenge. Serum collected on day 27 from SA-4-1BBL treated mice (25 μg on days 0 and 14) was injected intraveneously (i.v.) into naïve C57BL/6 mice (200 μl/animal) 24 hours prior to TC-1 challenge. FIG. 4D shows that B-cell depletion does not negate the prophylactic effect of SA-4-1BBL against tumor. C57BL/6 mice were pretreated twice, two weeks apart with SA-4-1BBL (25 μg/injection), followed by TC-1 (1×10⁵) challenge s.c. 2 weeks after the last SA-4-1BBL treatment. B cells were depleted using anti-CD20 antibody (clone 5D2; 200 μg/i.p. injection) 1 day before each SA-4-1BBL pretreatment. Data was analyzed using Kaplan-Meier Survival curve and log-rank statistical method. A P value ≤0.05 was considered significant.

FIG. 5A-I show that pretreatment with SA-4-1BBL increases the frequency of IFN-γ expressing memory-like CD4⁺ T cells and NK cells. C57BL/6 mice were treated s.c. twice, two weeks apart with SA-4-1BBL (n=4; 100 μg/injection) or agonistic 3H3 antibody (n=4). Animals were euthanized 3 days later to harvest injection-site draining lymph nodes for phenotyping using flow cytometry. FIG. 5A is a dot plot showing CD4⁺ and CD8⁺ T cells expressing CD44 and 4-1BB molecules. FIG. 5B and FIG. 5C show the frequency of memory-like CD44 expressing CD4⁺ and CD8⁺ T cells. FIG. 5D and FIG. 5E show the frequency of memory like CD4⁺ and CD8⁺ T cells expressing 4-1BB receptor. FIG. 5F is a dot plot showing CD4⁺ and CD8⁺ T cells and NK cells expressing CD44 and IFN-γ. Memory like CD4⁺ and CD8⁺ T cells expressing IL-2 (FIG. 5G) and IFN-γ (FIG. 5H), and NKT and NK cells expressing IFN-γ (FIG. 5I) are shown in FIG. 5G-I. Each data point is indicative of mean±SEM, with *P<0.05, **P<0.01, ***P<0.001; One way ANOVA with Bonferroni's Multiple Comparison Test.

FIG. 6A-F show that IFN-γ, CD4⁺ T, and NK cells are indispensable for the prophylactic anti-tumor efficacy of SA-4-1BBL. FIG. 6A shows the study design. FIG. 6B shows that blockade of IFN-γ results in tumor growth. C57BL/6 mice were pretreated s.c. twice with 100 μg SA-4-1BBL 2 weeks apart and challenged with TC-1 tumors 1 week later. One group received saline, while the other was treated with a blocking antibody against IFN-γ on days 0, 3, 14, 17, 20 as shown in FIG. 6A. Animals were monitored for tumor growth and euthanized when tumor size reached ≥12 mm in diameter or at 60-day experimental end-point. FIG. 6C shows depletion of NK cells results in tumor growth. C57BL/6 mice were pretreated s.c. twice, two weeks apart with SA-4-1BBL (25 μg/injection) followed by TC-1 challenge s.c. (1×10⁵) 2 weeks later. NK cells were depleted using an anti-NK1.1 antibody twice, one day before each SA-4-1BBL pretreatment or once one day before TC-1 challenge. D, Depletion of CD4⁺ T, but not CD8⁺ T cells, results in tumor growth. Same experimental design as in (C) except that an antibody to CD4 was used either one day prior to SA-4-1BBL immunizations or one day before TC-1 challenge. CD8⁺ T cells were depleted one day before SA-4-1BBL treatment. Data was analyzed using Kaplan-Meier Survival curve and log-rank statistical method. FIG. 6E and FIG. 6F show increased frequency of CD4⁺CD44⁺ T cells in lymphoid tissues of tumor-free mice. Mice were pretreated twice, two weeks apart with SA-4-1BBL followed by TC-1 tumor cell challenge 8 weeks later. Mice were then monitored for tumor growth for 60 days. Lymphocytes from spleens and draining lymph nodes of tumor-free mice were phenotyped for CD3⁺CD4⁺CD44⁺ and CD3⁺CD8⁺CD44⁺ cells using flow-cytometry. Each data point is indicative of mean±SD. P value ≤0.05 was considered significant using Student's t-test.

FIG. 7A-E show that SA-4-1BBL treatment prevents post-surgical recurrences. FIG. 7A shows the experimental design and TC-1 tumor relapse curves. C57BL/6 mice bearing TC-1 tumors (˜4 mm in diameter) were subjected to surgery to remove the tumor. Two days later, mice were treated s.c. with SA-4-1BBL (25 μg/injection) twice, two weeks apart, and monitored for tumor recurrence. FIG. 7B shows results from an experiment the same as in FIG. 7A except that 3LL-huMUC1 tumor cells were used. Mice that underwent surgical resection of the tumor without SA-4-1BBL treatment were used as controls. FIG. 7C, SA-4-1BBL therapy is associated with long-term immunological memory. Mice without tumor in (A) were re-challenged s.c. with TC-1 cells (1×10⁵) and monitored for additional 60 days for recurrences. Data was analyzed using Kaplan-Meier Survival curve and log-rank statistical method. A P value ≤0.05 was considered significant. FIG. 7D shows the experimental design for B16-F10 tumor relapse experiments. FIG. 7E shows the B16-F10 tumor relapse curves. C57BL/6 mice bearing B16-F10 melanoma tumors (4 mm in diameter) were subjected to surgery to remove the tumor. Two days later, mice were treated subcutaneously either with SA-4-1BBL (100 μg/injection) twice, 2 weeks apart, and monitored for tumor recurrence. Mice that underwent surgical resection of the tumor without any treatment were used as controls. Tumor measurements were recorded twice weekly using calipers and mice were euthanized when tumor ulcerated or size reached 12 mm in diameter. The graph was plotted as tumor incidence and data were analyzed using unpaired t test (two tailed) with a p value ≤0.05 was considered significant.

FIG. 8A-B set forth the amino acid sequences of representative SA-4-1BLL fusion proteins: FIG. 8A shows the amino acid sequences of core streptavidin (CSA) with the extracellular domain of murine 4-1BBL and FIG. 8B shows the amino acid sequences of core streptavidin (CSA) with the extracellular domain of human 4-1BBL. The core streptavidin sequence is underlined.

FIG. 9A-D shows that SA-4-1BBL is a bona fide novel anti-tumor immunomodulator that has preventive efficacy against various tumor types. FIG. 9A shows the experimental design. FIG. 9B shows results when C57BL/6 mice were pretreated s.c in the left flank with 100 μg of SA-4-1BBL protein twice, 2 weeks apart, and challenged 2 weeks later s.c. in the left flank with 1×10⁵ B16-F10 melanoma tumor cells. BALB/c mice were pretreated s.c. in the left flank with 100 μg of SA-4-1BBL protein or 100 μg of 3H3 agonistic antibody, 2 weeks apart, and challenged 2 weeks later s.c. in the left flank with 1×10⁶ A20 lymphoma (FIG. 9C) or 5×10⁴ 4T1 breast cancer (FIG. 9D) tumor cells as shown in FIG. 9C-9D. Tumor measurements were recorded twice weekly using calipers and mice were euthanized when tumor ulcerated or size reached 12 mm in diameter. Data were analyzed using Kaplan-Meier survival curve and log-rank (Mantel-Cox) statistical method. A P value ≤0.05 was considered significant.

### DETAILED DESCRIPTION

Described herein are methods for treating and preventing cancer. In particular, described herein are methods using SA-4-1BBL to treat, prevent or reduce the risk of cancer, treat, prevent, or reduce the risk of tumorigenesis, and treat, prevent, or reduce the risk of tumor recurrence, as well as SA-4-1-BBL for use in such methods.

For the purposes of the present application, the following terms have these definitions:

As used herein “a” or “an” means one or more, unless specifically indicated to mean only one.

“Administration” and similar terms as used herein encompass all suitable means of providing a substance to a subject, including subcutaneously, intramuscularly, and intrathecally.

“Antigen” is used herein without limitation, and includes proteins, lipids, sugars, nucleic acids, chemical moieties, and other moieties that induce an immune response. Antigens associated with a cancer or tumor include antigens that are part of the tumor cells.

“Subject” as used herein includes any mammal, including equine, ovine, caprine, bovine, porcine, canine, feline and primate species. In any embodiments described herein, the subject is human. “Tumor” as used herein includes solid and non-solid tumors (such as leukemia), and different stages of tumor development from pre-cancerous lesions and benign tumors, to cancerous, malignant and metastatic tumors.

“Cancer” as used herein refers to a disease or condition of abnormal cell growth characteristics. A “cancer” as used herein may be a sarcoma, a carcinoma, a lymphoid, or a leukemia. The cancer may arise in any tissue and may be, for example, a skin cancer such as melanoma, squamous carcinoma or basal cell carcinoma, a brain cancer, a head and neck cancer, a lung cancer, a liver cancer, a pancreatic cancer, a colon cancer, an eye cancer such as retinoblastoma, or kidney cancer such as Wilm's tumor, and so on. “Preneoplastic or early neoplastic lesions” as used herein refers to abnormal tissue growth that has a higher risk than the normal surrounding tissue of developing into a malignant lesion or a cancer.

“Treating” or “treatment” of a cancer in a subject as used herein refers to inhibiting the abnormal cell growth or arresting its development and/or ameliorating or causing regression of the abnormal cell growth (“shrinking the tumor”), and includes diminishment of the extent of a cancer, stabilized (i.e., not worsening) state of a cancer, delay or slowing of cancer progression, amelioration or palliation of the cancer, and cancer remission (whether partial or total). “Preventing” or “prevention” of a cancer or tumor refers to preventing or reducing the risks of abnormal cell growth, including in a subject who is at risk of developing a tumor or cancer. A subject may be at risk of developing a cancer or tumor by having one or more of any number of risk factors, including genetic risk factors, lifestyle risk factors, environmental risk factors, and other disease-associated risk factors. Additionally or alternatively, a subject may be at risk of developing cancer or tumors if the subject has previously been treated for a cancer or tumor.

As used herein the term “effective amount” refers to an amount that is sufficient to achieve a desired clinical response in accordance with the purpose of the treatment. An effective amount can be administered in one or more administrations, applications, or dosages, and may vary for any particular patient depending upon a variety of factors, including age, body weight, general health, tumor or cancer status, etc. The invention described herein stems from the surprising and unexpected discovery that SA-4-1BBL used as a single agent without any antigen (e.g., not as an adjuvant and not in conjunction with a tumor-associated antigen), protected naive mice against tumor challenge, and the further surprising and unexpected discovery that this effect was not tumor specific. While not wanting to be bound by theory, we believe this effect operates through the expansion of memory like CD4+ T cells that communicate with NK cells as effectors. Our results also indicate that IFN-γ is required for tumor eradication by SA-4-1BBL. We also determined that SA-4-1BBL monotherapy prevented post-surgical tumor growth. Together, these results support our characterization of SA-4-1BBL as a unique immunostimulator that can both prevent and treat cancer.

Costimulation through 4-1BB (CD137) receptor generates robust CD8⁺ T effector and memory responses, which serves as an impetus for targeting this pathway for cancer immunotherapy. The only known ligand, 4-1BBL, is a trimeric transmembrane protein that has no costimulatory activity as a soluble molecule. Thus, agonistic antibodies to the receptor have been used for cancer immunotherapy in preclinical models and are currently being evaluated in the clinic.

We herein report surprising and unexpected findings that treatment with SA-4-1BBL alone protects mice against subsequent tumor challenge. This is a unique feature of the SA-4-1BBL molecule because an agonistic 4-1BB antibody did not protect mice against tumor challenge. This prophylactic effect of SA-4-1BBL was long-lasting (>8 weeks), tumor type-independent, and involved both CD4⁺ T and NK cells as well as IFN-γ. Moreover, treatment with SA-4-1BBL after surgical removal of tumors resulted in control of relapses. That is, SA-4-1BBL as a single agent also had robust efficacy in controlling postsurgical recurrences. To our knowledge, this is the first study to demonstrate that an immune checkpoint stimulator such as 4-1BBL can prime or train the immune system for cancer prevention. We therefore disclose SA-4-1BBL an immune modulator with distinct immune functions as compared to agonistic 4-1BB antibody, with broad implications and applications for cancer immunoprevention and therapy.

In particular, we herein report surprising and unexpected findings that pretreatment with SA-4-1BBL as a robust agonist of 4-1BB costimulatory pathway protects mice against tumor challenge. Prophylactic efficacy is not restricted to a particular tumor type and is long-lasting. In contrast, an agonistic antibody to the 4-1BB receptor did not alter tumor growth. Prophylactic efficacy of SA-4-1BBL appears to involve a cross-communication between CD4⁺CD44⁺“memory-like” T and NK cells and appears to require IFN-γ. Mechanistically, SA-4-1BBL significantly expanded IFN-γ expressing pre-existing memory-like CD44⁺CD4⁺ T cells and NK cells in naïve mice as compared to the agonistic antibody. In vivo blockade of IFN-γ or depletion of CD4⁺ T or NK cells, but not CD8⁺ T or B cells, abrogated the SA-4-1BBL cancer immunoprevention efficacy. To the best of our knowledge, this is the first demonstration that an immunomodulator without an antigen primes the immune system for the prevention of tumor growth.

This work highlights surprising and unexpected features of SA-4-1BBL as a novel immunomodulator with implications for cancer immunoprevention and therapy.

Immunotherapy for cancer has gained significant impetus precipitated by the remarkable clinical therapeutic efficacy of immune checkpoint inhibitors. Costimulatory receptors that initiate and sustain effector and memory immune responses represent the next potential targets for cancer immunotherapy. Among the costimulatory checkpoint receptors, 4-1BB is upregulated on T cells following activation and signaling through this pathway plays a paramount role in promoting T cell survival, expansion, acquisition of effector function, and long-term memory. The importance of this pathway for cancer immunotherapy has already been demonstrated by incorporation of 4-1BB signaling into the CAR T-cell technology and the use of agonistic antibodies to the receptor as mono or combination therapies in preclinical as well as clinical settings. However, a major drawback for the use of agonistic antibodies has been significant toxicity observed both in the preclinical and clinic settings. We, therefore, hypothesized that toxicity might not be an inherent feature of 4-1BB signaling, but rather an adverse effect of agonistic antibodies. Tumor-associated antigen-based subunit vaccines adjuvanted with SA-4-1BBL showed therapeutic efficacy in various preclinical cancer models without toxicity associated with agonistic 4-1BB antibodies.

The initial intent of this study was to test if streptavidin portion of SA-4-1BBL as a foreign antigen is immunogenic and assess the impact of anti-streptavidin antibodies on the therapeutic efficacy of SA-4-1BBL-adjuvanted cancer vaccines. Using the well-established TC-1 tumor model, we demonstrated that pretreatment of naïve mice with SA-4-1BBL as a single agent generated both humoral and cellular immune responses to the streptavidin. However, anti-streptavidin antibodies did not block the costimulatory function of SA-4-1BBL in an in vitro T cell proliferation assay. Vaccination of mice with preexisting high titers of anti-streptavidin antibodies with a subunit vaccine containing a synthetic peptide representing CD8⁺ T cell epitope for E7 adjuvanted with SA-4-1BBL generated a therapeutic response, resulting in eradication of TC-1 tumor in all mice. TC-1 cells express the E7 oncoprotein from HPV-16, and are commonly used as a model for human tumors infected with HPV-16. These in vitro and in vivo findings demonstrating lack of a negative impact of anti-streptavidin antibodies on the costimulatory function of SA-4-1BBL are consistent with the structural design of this molecule. Streptavidin is linked to the C-terminus of the extracellular domain of murine 4-1BBL through a linker that provides flexibility and allows spatial separation of both molecules. It is, therefore, not surprising that anti-streptavidin antibodies do not interfere with the T cell costimulatory function of SA-4-1BBL.

Treatment with SA-4-1BBL as a single agent (monotherapy) conferred protection against subsequent tumor challenge in mice. This highly novel and unexpected prophylactic efficacy was dose-dependent and effective against five different tumor types. The tumor preventive effect required 3 weeks to fully evolve following first SA-4-1BBL treatment and lasted for more than 8 weeks. In marked contrast, pretreatment with an agonistic 4-1BB antibody did not impact tumor growth. The immunogenicity of streptavidin in SA-4-1BBL was not responsible for the observed protective effect against tumors, as treatment with the agonistic antibody combined with streptavidin also failed to generate a tumor-preventive response. Importantly, SA-4-1BBL and the agonistic 3H3 antibody had opposite effects on the generation of streptavidin antibodies; SA-4-1BBL generating high titers, while the agonistic 3H3 antibody blocking such a response. This observation is consistent with a previous study reporting 4-1BB agonistic antibodies inducing anergy in CD4⁺ T cells that resulted in the blockade of humoral responses. However, lack of a humoral response to streptavidin was not responsible for the inability of agonistic antibody in protecting mice against tumor challenge. Passive transfer of serum with high titers of streptavidin antibodies did not prevent naïve mice against tumor challenge. Consistent with the lack of a humoral response, B cell depletion did not negate the prophylactic efficacy of SA-4-1BBL against tumor challenge.

We demonstrated that a subpopulation of CD4⁺ T cells in naïve mice express both CD44 memory marker and 4-1BB receptor on their surface and respond to SA-4-1BBL treatment by significant expansion. The presence of “memory-like” CD4⁺ T cells have previously been reported by others. Although, the exact nature of these cells remains to be fully elucidated, they may represent cells that had responded to pathogenic/environmental antigens or activated due to physiological homeostatic proliferation. Consistent with a previously published study demonstrating that agonistic antibodies to 4-1BB deliver antigen-independent growth signal in T cells having memory-like phenotype in naïve mice, the agonistic 3H3 antibody used in our study also expanded memory-like T cells. However, SA-4-1BBL differed from the agonistic antibody by significantly increasing the number of CD4⁺ memory T cells and NK cells producing IFN-γ. Treatment with SA-4-1BBL also increased the frequency of CD4⁺CD44⁺ T cells expressing IL-2 as compared with naïve, significant, and 3H3 treated mice, trending towards significance.

Cross-communication between CD4⁺ T cells and NK cells has been reported in various infection and tumor models and orchestrated by IL-2 and IFN-γ. IL-2 produced by antigen-activated CD4⁺ T cells plays an important role in the activation, expansion, and production of cytokines, particularly IFN-γ by NK cells. Once IFN-γ is produced, it drives T cell responses towards a Th1 response, which is critical for tumor eradication. Consistent with these previously published studies, both CD4⁺ T and NK cells as well as IFN-γ appeared to be required for SA-4-1BBL-generated cancer preventive effect. Depletion of either cell population at the priming or effector phases or blockade of IFN-γ in vivo resulted in the abrogation of SA-4-1BBL conferred prophylactic effect against tumor. The importance of CD4⁺ T and NK cell interplay in immune responses against infections and cancer has previously been reported. Antigen-primed T cells were shown to play a requisite role for the activation of NK cells and production of IFN-γ in a Leishmania major infection preclinical model, primarily through the secretion of IL-2. Collaboration between CD4⁺ T cells and NK cells was also shown in a B16 melanoma preclinical model lacking CD4⁺ T cells and in patients with HIV-1 viral infection that compromises CD4⁺ T cell number and IL-2 production, resulting in NK cell anergy and irresponsiveness to infection. Immunization with an HIV-1 subunit vaccine resulted in increased IL-2 production by antigen-specific CD4⁺ T cells and IFN-γ by NK cells. Depletion of CD4⁺ T cells, which we have shown to play a critical role in SA-4-1BBL-adjuvanted subunit vaccines, had no impact on the preventive effect of SA-4-1BBL, which is consistent with the lack of sustained immune memory in this model.

In addition to its unexpected preventive effect, monotherapy with SA-4-1BBL was effective in preventing post-surgical tumor recurrences. It has been shown that although spontaneous T cell responses are inherently generated with tumor growth, these cells are not truly functional and effective. SA-4-1BBL treatment post-surgical treatment could be targeting these T cells that are activated against tumor antigens and express high levels of the 4-1BB receptor. Indeed, 4-1BB is used as a bona fide marker to sort tumor-specific T cells for ex vivo expansion and adoptive cell therapy. Thus, the engagement of SA-4-1BBL as monotherapy with its receptor 4-1BB serves as a convenient and effective way of expanding T cells that are primed by tumor neoantigens, leading to the acquisition of effector functions and controlling recurrences. This notion is consistent with our observations that SA-4-1BBL-mediated control of tumor recurrences was associated with long-term memory. Our findings are also consistent with a previously published report demonstrating that agonistic 4-1BB antibodies protect mice against post-surgical tumor challenge by expanding tumor-primed CD4⁺ T cells.

In conclusion, our data demonstrate unique and unexpected immunomodulatory features of SA-4-1BBL that bridge innate and adaptive immune responses with both preventive as well as therapeutic efficacy against cancer. The tumor type-independent protection conferred by SA-4-1BBL is significant with important clinical implications for primary and secondary cancer prevention modalities. This agent may have utility for the treatment of individuals at high risk for tumorigeneses, such as patients with lifestyle risk factors, environmental risk factors, chronic liver diseases, individuals with hereditary mutations in p53 or breast cancer (BRCA1 and BRCA2) genes, those with a DNA repair deficiency, and those affected by specific cancer risks, such as preneoplastic/early neoplastic lesions. For example, a subject may have a history of hereditary BRCA1 and/or BRCA2 mutations (breast cancer), Lynch Syndrome (hereditary non-polyps colorectal cancer), Cowden Syndrome (mutation in PTEN gene); or be infected with HPV (a risk factor for cervical and head and neck cancers). The safety profile of SA-4-1BBL and its cancer immunoprevention attributes, both of which are not shared by agonistic antibodies to the 4-1BB receptors, highlight its potential for cancer immunoprevention and therapy.

Immune co-stimulatory molecules are involved in the natural interaction between naive T cells and antigen presenting cells, which results in their reciprocal activation and prompts the expression of various cell surface ligands and receptors, and soluble proteins that contribute to the initiation, maintenance, and long-term memory of the immune response. At least three signals are required for the initial activation of naive T cells. Signal 1 is generated by interactions between a T cell receptor (TCR) and a nominal peptide presented by major histocompatibility complex (MEW) molecules on the surface of professional APC, such as dendritic cells (DC). Signal 2 can be mediated by several different molecules and is important to a sustained immune response. Signal 3 is transduced via cytokines elaborated by activated T cells and APC and is important to the maintenance of effector immune responses.

### SA-4-1-BBL

As noted above, SA-4-1BBL is a fusion protein of the extracellular portion of 4-1BBL to the C-terminus of a modified form of core streptavidin (SA or CSA). As discussed above, the SA-4-1BBL molecule exists as tetramers/oligomers.

4-1BBL (also known as 4-BB-L, 4-BB ligand, TNFSF9, ILA ligand) is a type II protein expressed on activated B cells, macrophages, and DC two to three days following activation. 4-1BB/4-1BBL interactions also transduce Signal 2 to CD8⁺ T cells in a CD28-independent manner and stimulate them to produce cytokines, expand, and acquire effector functions. 4-1BBL contains 254 amino acids (26624 Da). See Alderson et al. Eur J Immunol. 1994 September; 24(9):2219-27. The full amino acid sequence of human 4-1BBL can be found under accession no. P41273 in the Swiss-Prot database. 4-1BBL is a type II glycoprotein with residues 1-28 forming a potential cytoplasmic domain, residues 29-49 forming a single predicted transmembrane domain, residues 50-254 forming a potential extracellular domain, and residues 35-41 representing a poly-Leu stretch. The nucleotide sequence in humans encoding the 4-1BBL can be found in GenBank accession no. NM003811. Residues 50-254 of 4-1BBL or fragments thereof that can bind to its cognate receptor 4-1BB.

Core streptavidin (“SA” or “CSA”) is a truncated version of the full-length streptavidin polypeptide which may include streptavidin residues 13-138, 14-138, 13-139 or 14-139. The nucleic acid sequences encoding streptavidin and avidin and the streptavidin and avidin amino acid sequences can be found, for example, in GenBank Accession Nos. X65082; X03591; NM205320; X05343; 221611; and Z21554.

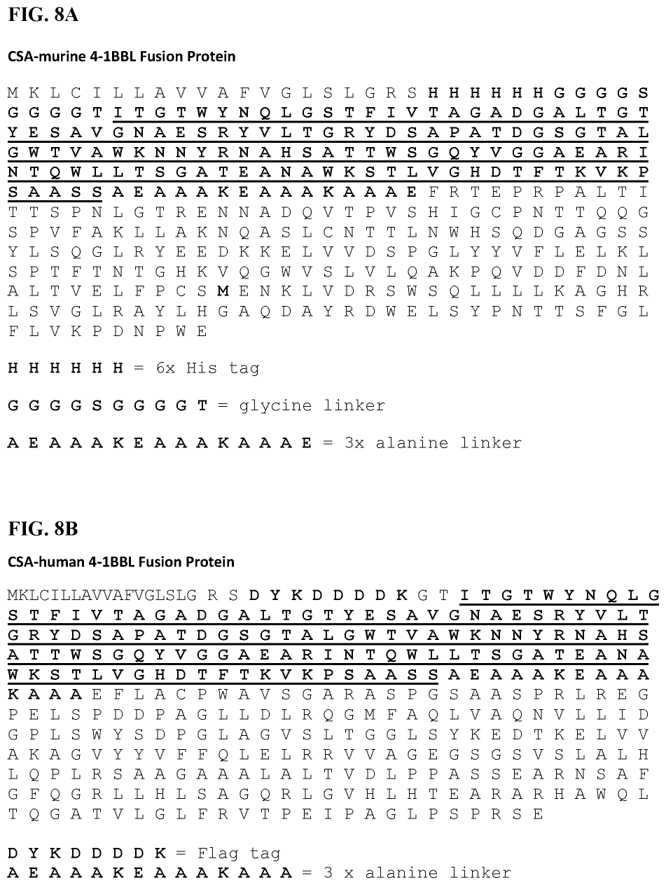

The amino acid sequences of representative SA-4-1-BBL fusion proteins based on murine or human 4-1-BBL are set forth in FIG. 8A-8B. As shown in the figure, the fusion proteins may include one or more of a 6×His tag, a Flag tag, a glycine linker, a 3× alanine linker, or any other moiety that may facilitate expression and/or purification, such as maltose binding protein, glutathione S-transferase, intein-chitin binding domain, and the like. In any of these embodiments, the tag or moiety may be joined to SA-4-1BBL at the N-terminus of the SA-4-1-BBL construct. Alternatively, the tag or moiety may be joined to SA-4-1BBL at the C-terminus of the SA-4-1-BBL construct. In accordance with any embodiments, the SA-4-1-BBL may have the amino acid sequence set forth in FIG. 8A or FIG. 8B.

### Compositions

Also provided herein are compositions comprising a SA-4-1-BBL conjugate as described herein and a carrier, and pharmaceutical compositions comprising a SA-4-1-BBL conjugate as described herein same a pharmaceutically acceptable carrier. As used herein, a “carrier” refers to a material that can be used as a vehicle or diluent for the conjugate because it does not react with and is compatible with the conjugate. A pharmaceutically acceptable carrier is a material that can be used as a vehicle or diluent for the conjugate because the material is medically acceptable for the intended subject and route of administration and does not react with and is compatible with the conjugate. A pharmaceutically acceptable carrier can contain conventional pharmaceutical additives well known in the art, including preservatives, pH adjusting agents, tonicity agents, and the like. In some embodiments, the pharmaceutically acceptable carrier is suitable for administration by intravenous, subcutaneous, or intraperitoneal injection.

The carrier may provide targeted delivery and/or controlled release of the conjugate. For example, the carrier may include micelles, liposomes, nanoparticles, or emulsions comprising the SA-4-1-BBL conjugate.

The composition may comprise the SA-4-1-BBL conjugate at any suitable concentration at which the conjugate is stable against precipitation and provides a therapeutically effective amount of conjugate in therapeutically acceptable volume for the intended route of administration.

### Methods

In accordance with the methods described herein, a therapeutically effective amount of a SA-4-1-BBL conjugate as described herein is administered to a subject in need thereof, such as a subject in need of the prevention of (or reducing the risk of) or treatment of a cancer, a subject in need of reducing the risk of tumorigeneses, or a subject in need of reducing the risk of tumor recurrence, such as after cancer or tumor treatment, such a subject in need of reducing the risk of post-surgery, post-chemotherapy, or post-irradiation treatment tumor recurrence, such as a subject who has undergone surgical remover of tumor cells, chemotherapy or cancer irradiation treatment. The methods described herein are monotherapy methods. As used herein, “monotherapy methods” refers to methods that do not include administering to the subject an antigen associated with the cancer or tumor. Thus, in accordance with the methods described herein, SA-4-1-BBL conjugate is not used as an adjuvant or administered with a tumor-associated or cancer-associated antigen.

In accordance with any of the methods or uses described herein, the subject may at risk of developing cancer due to lifestyle risk factors, such as obesity, smoking, and/or alcohol use, or exposure to environmental risk factors, such as asbestos, UV radiation, ionizing radiation, etc.

In specific cases, the subjects being treated may be at high risk for tumorigeneses, such as subjects with chronic liver diseases, individuals with hereditary mutations in p53 or breast cancer (BRCA1 and BRCA2) genes, those with a DNA repair deficiency and those affected by specific cancer risks, such as preneoplastic/early neoplastic lesions. A subject may have a history of hereditary BRCA1 and/or BRCA2 mutations (breast cancer), Lynch Syndrome (hereditary non-polyps colorectal cancer), Cowden Syndrome (mutation in PTEN gene), and/or be infected with HPV (a risk factor for cervical and head and neck cancers). A subject may suffer from, be at risk of developing, or have been treated for any type of cancer, including melanoma, lymphoma, lung cancer, or breast cancer.

The subject may be at risk of tumor recurrence due to, for example having undergone surgical remover of tumor cells, chemotherapy or cancer irradiation treatment.

In accordance with specific embodiments, the methods described herein provide a prophylactic approach that protects high risk individuals from non-virally-associated cancers that will significantly reduce cancer burden.

The SA-4-1-BBL conjugate may be administered by any suitable route of administration, such as intravenously, subcutaneously, or intraperitoneally. The SA-4-1-BBL conjugate may be administered at any dose that provides a therapeutic effect. The dose may be from about 0.001 to about 100 mg/kg, including from about 0.005 mg/kg to about 50 mg/kg, from about 0.01 mg/kg to about 10 mg/kg, from about 0.05 mg/kg to about 5 mg/kg, and from about 0.1 mg/kg to about 1 mg/kg. In some embodiments, SA-4-1-BBL is administered at a dose of about 0.001 mg/kg, about 0.005 mg/kg, about 0.01 mg/kg, about 0.05 mg/kg, about 0.1 mg/kg, about 0.5 mg/kg, about 1.0 mg/kg, about 5 mg/kg, about 10 mg/kg, about 50 mg/kg, or about 100 mg/kg.

The methods may comprise administering SA-4-1-BBL according to any therapeutically effective dosing regimen, and may continue until the desired clinical effect is achieved, including for weeks, months, or years. The conjugate may be administered daily, on alternate days, twice weekly, weekly, twice a month, or once a month. In specific embodiments, the conjugate is administered twice, two weeks apart, optionally followed by a rest period of, for example, 2-6 months, after which the dosing regimen maybe repeated until the desired clinical effect is achieved. The following examples illustrate the invention in more detail, and are not intended to limit the scope of the invention in any respect.

### EXAMPLES

### Materials and Methods

### Mice

C57BL/6 mice were purchased from The Jackson Laboratory. Mice were bred and cared for in a University of Louisville specific pathogen-free animal facility in accordance with NIH guidelines. All animal procedures were conducted under protocols approved by Institutional Animal Care and Use Committee at the University of Louisville.

### Antibodies, Recombinant Proteins, and Cell Lines

Fluorescent-conjugated antibodies to various cell surface markers were obtained commercially including: α-CD3-V500 (BD Horizon 560771); α-CD4-Alexa700 (BD Pharmingen 557956); α-CD8-APC-Cy7 (BD Pharmingen 557654); α-NK1.1-PE (BD Pharmingen 553165); α-CD19-APC (BD Pharmingen 550992); α-CD19-PE-Cy7 (eBioscience 25-0193-82); α-CD44-APC (eBioscience 17-0441-83); α-CD62L-PerCp-Cy5.5 (eBioscience 45-0621-82); α-CD69-FITC (BD Pharmingen 553236); α-4-1BB-PE (eBioscience 12-1371-83).

Anti-4-1BB agonistic antibody (clone 3H3) was produced in our laboratory. Antibodies used intraperitoneally (i.p.) for the depletion of specific immune cells include CD4 (clone GK1.5, 500 μg/injection), NK1.1 (clone PK136, 500 μg/injection), CD8 (clone 53.6.72, 500 μg/injection), and CD20 (clone 5D2, 200 μg/injection). IFN-γ was blocked in vivo by i.p. injection of 200 μg of an anti-IFN-γ antibody (clone XMG1.2, BioXcell) on days 0, 3, 14, 17, 20 with respect to first SA-41BBL treatment. SA-4-1BBL and streptavidin proteins were produced in our laboratory according to standard protocols as previously reported. TC-1 and Lewis lung carcinoma (LLC) tumor cell lines were obtained and maintained according to American Type Culture Collection (ATCC).

### SA-4-1BBL Treatment and Tumor Challenge

Mice were treated s.c. with SA-4-1BBL at the indicated doses once or twice two weeks apart as specified. Mice were challenged s.c. in the left back flank with 1×10⁵ live TC-1, LLC, or huMUC1-LLC tumor cell lines as indicated. Selected groups were vaccinated 6 days post-tumor challenge with 50 μg of HPV E7 peptide 1 (P1, RAHYNIVTF) serving as the dominant E7 epitope for CD8⁺ T cells adjuvanted with 25 μg SA-4-1BBL protein. Animals were monitored for tumor growth, and tumors were measured twice a week using calipers. Animals were euthanized at a 60-day experimental end-point or when tumors ulcerated or reached a size of ˜12 mm in diameter.

To test the therapeutic efficacy of SA-4-1BBL as monotherapy, TC-1 or 3LL-huMUC1 tumors of ˜4 mm in diameter were surgically removed under sterile conditions and avertin anesthesia (250 mg/kg). After 48 hours of recovery period, animals were treated s.c. with SA-4-1BBL (25 μg/injection) twice, two weeks apart. Animals without SA-4-1BBL treatment served as controls and were monitored for tumor relapse.

### Anti-Streptavidin Antibody Titers

Sera collected at the indicated times from control and treatment groups were assessed for anti-streptavidin antibodies using ELISA. Briefly, 96-well flat-bottom plates were coated with SA-4-1BBL (50 ng/well) or control streptavidin (50 ng/well) proteins in sterile PBS and incubated overnight at 4° C. Wells were then washed three times with the wash buffer (PBS/Tween-20) then incubated with a nonfat milk blocking buffer for 1 h to block nonspecific binding. After washing the plate three times with the wash buffer, the wells were incubated with serial dilutions of sera at room temperature for 1.5 h. After several washes, the wells were incubated with a secondary antibody conjugated to horseradish peroxidase (HRP) for 1 h. Plates were then incubated for 30 min with TMB substrate (BD Biosciences, Cat #555214) and read on Wallac Victor 1420 Multilabel microplate reader at 450 nm.

### Passive Serum Transfer

Mice were treated s.c. twice with SA-4-1BBL (25 μg/treatment) two weeks apart and serum was collected 27 days after the initial treatment. Serum was assessed for antibody titers against streptavidin and then injected i.v. into C57BL/6 mice (200 μl/animal) 24 hours prior to TC-1 tumor challenge s.c. (1×10⁵ cells).

### SA-4-1BBL T Cell Costimulation In Vitro Assay

C57BL/6 splenocytes (2×10⁵ cells/well) were cultured in 96-well U-bottom plates and stimulated with a suboptimal dose of an agonistic antibody to CD3 (0.25 μg/ml). Cultures were then supplemented with various doses of SA-4-1BBL preincubated at room temperature for 1 hour in naïve serum or serum with positive antibody titers against SA. Cultures were then incubated for 48 hours and pulsed with [³H]-thymidine for an additional 16 hours. Plates were harvested with Tomtec Cell Harvester, and DNA-associated radioactivity was measured using a Beta plate counter and graphed as counts per minute (CPM).

### Flow Cytometry and Phenotyping

Lymphocytes harvested from the spleen and injection site-draining lymph nodes of naïve or various treatment groups were stained with fluorescent-conjugated antibodies to various cell surface and intracellular markers. Cells were analyzed using multiparameter LSRII flow cytometry (BD Biosciences) by gating on live cells. Cell percentages and absolute numbers were calculated and reported.

### Statistics

Statistics were performed with GraphPad Prism 6 software (La Jolla, Calif.). Survival was assessed using Kaplan-Meier method and log-rank test. Student's t-tests were used to compare differences between two individual groups. Where indicated, one way ANOVA was applied as well. P values of ≤0.05 were considered statistically significant.

### Results

### SA-4-1BBL as a Monotherapy Protects Mice Against Tumor Challenge

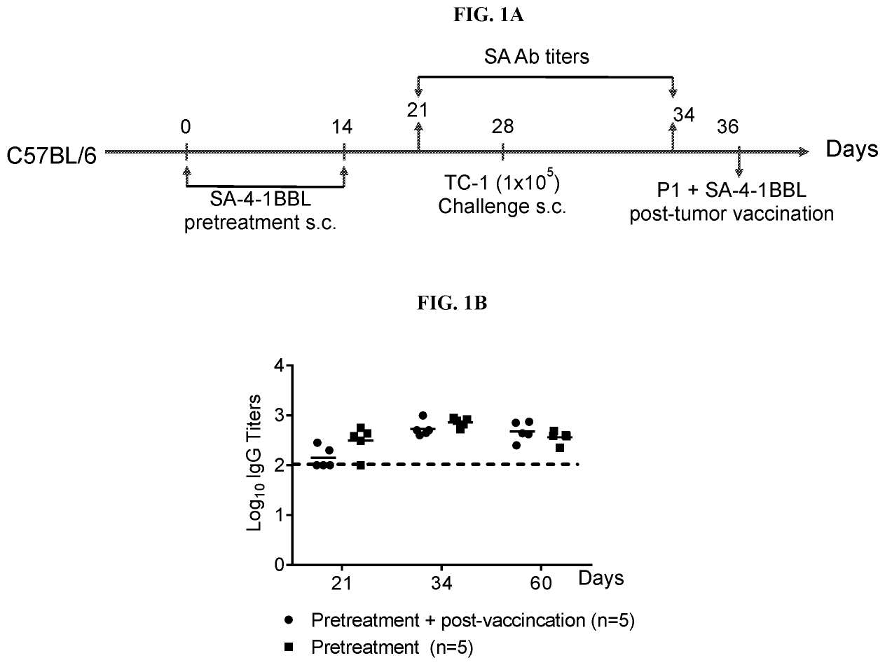

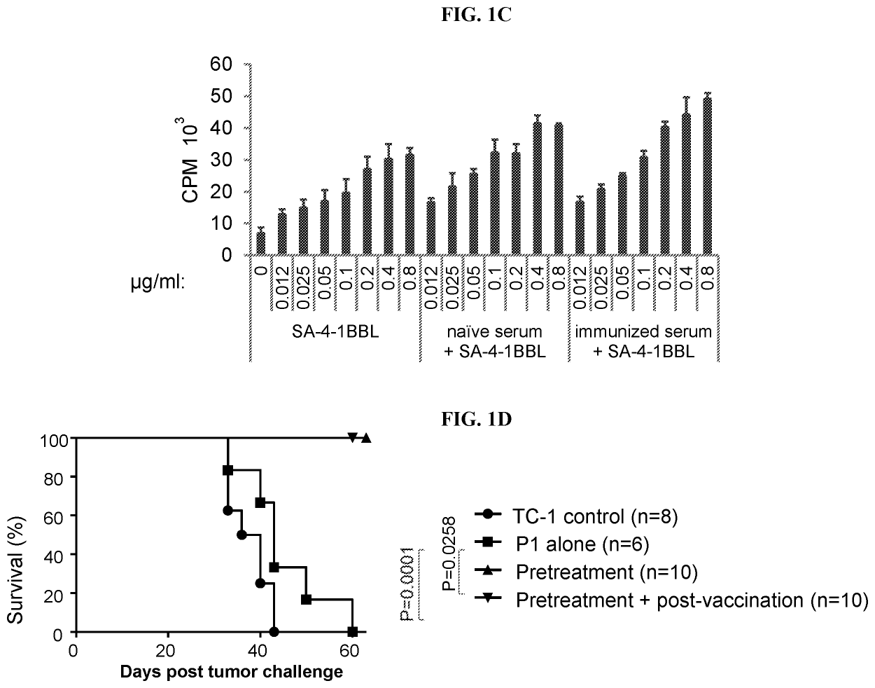

We previously demonstrated that a subunit vaccine containing a synthetic peptide (P1) representing the dominant CD8⁺ T cell epitope for human papilloma virus (HPV) E7 protein adjuvanted with SA-4-1BBL had therapeutic efficacy against HPV TC-1 tumor model in C57BL/6 mice. Streptavidin as a bacterial protein has the potential to generate humoral immune responses that may negatively impact the efficacy of SA-4-1BBL-adjuvanted vaccines. To assess this possibility, mice were pretreated twice with SA-4-1BBL protein alone (25 μg/injection) two weeks apart, followed by TC-1 tumor challenge and vaccination as schematically depicted in FIG. 1A. Treatment with SA-4-1BBL protein generated both humoral (FIG. 1B) and cellular (data not shown) immune responses against streptavidin. To assess if anti-streptavidin antibodies impact the function of SA-4-1BBL, the protein was preincubated with naïve or immune serum and then tested for T-cell costimulatory activity in vitro. SA-4-1BBL protein pre-incubated with serum containing high titers of anti-streptavidin antibodies had a similar costimulatory activity in driving T cell proliferation as SA-4-1BBL preincubated with naïve serum (FIG. 1C).

Consistent with the in vitro data, pretreatment with SA-4-1BBL protein did not alter the therapeutic efficacy of the subunit vaccine, as all mice in this group remained tumor-free for a 60-day observation period (FIG. 1D). Surprisingly, pretreatment with SA-4-1BBL alone also protected all mice against TC-1 tumor challenge (FIG. 1D). Together, these data demonstrate that the immunogenicity of streptavidin does not negatively impact the therapeutic efficacy of SA-4-1BBL-adjuvanted vaccine and that SA-4-1BBL as a single agent protects mice against tumor challenge, a highly surprising and unexpected finding.

### SA-4-1BBL Generates a Rapid and Lengthy Window of Protection Against Tumor

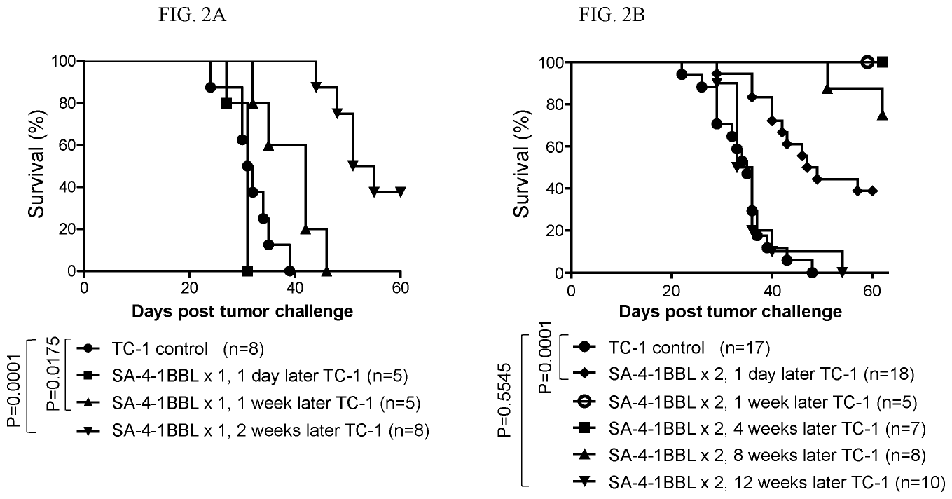

We next assessed the kinetics of SA-4-1BBL-conferred protection against tumor. A single treatment with SA-4-1BBL followed by TC-1 tumor challenge one day later did not result in protection, as all mice developed tumor at a rate similar to untreated, control animals (FIG. 2A). Delaying the tumor challenge by one week resulted in significant retardation in tumor growth (p<0.02), but all mice eventually expired from the tumor burden (FIG. 2A). The SA-4-1BBL mediated protective response was further improved when tumor challenge was delayed by two weeks, resulting in the survival of ˜40% of mice during a 60-day observation period.

Treatment with SA-4-1BBL twice, two weeks apart improved tumor response kinetics, efficacy, and duration. Forty percent of mice challenged with TC-1 tumor one day after the second SA-4-1BBL treatment survived over an observation period of 60 days (FIG. 2B). Delaying the time of tumor challenge by 1, 2, or 4 weeks resulted in complete protection, as all mice in these three groups survived by the 60-day experimental end-point (FIGS. 2B, 1D). Importantly, the tumor-protective effect was long-lasting as 80% of mice challenged with tumor eight weeks post SA-4-1BBL treatment survived by the 60-day experimental end-point. However, the SA-4-1BBL conferred protective effect was lost when tumor challenge was delayed by twelve weeks (FIG. 2B). These data demonstrate that the SA-4-1BBL-induced protective response is rapid, evolves fully within three weeks, and lasts more than eight weeks.

SA-4-1BBL-Generated Protection is Tumor-Type Independent and does not Evolve into a Long-Lasting Immune Memory

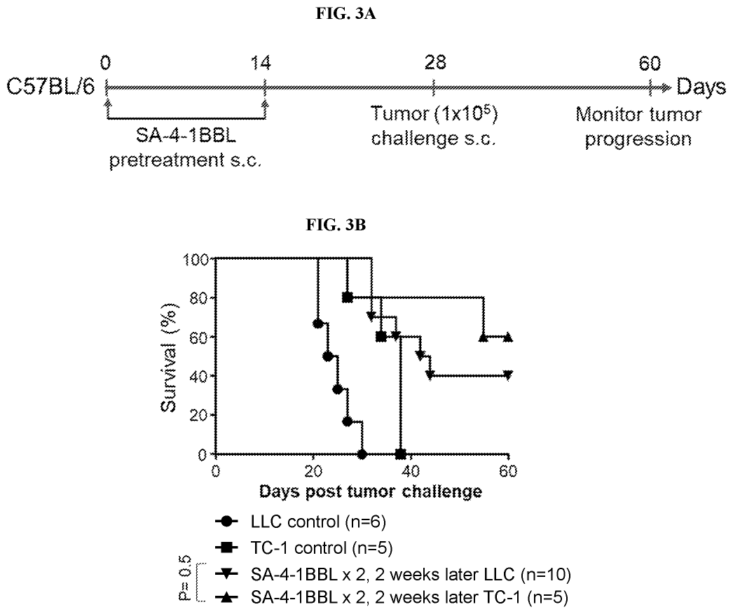

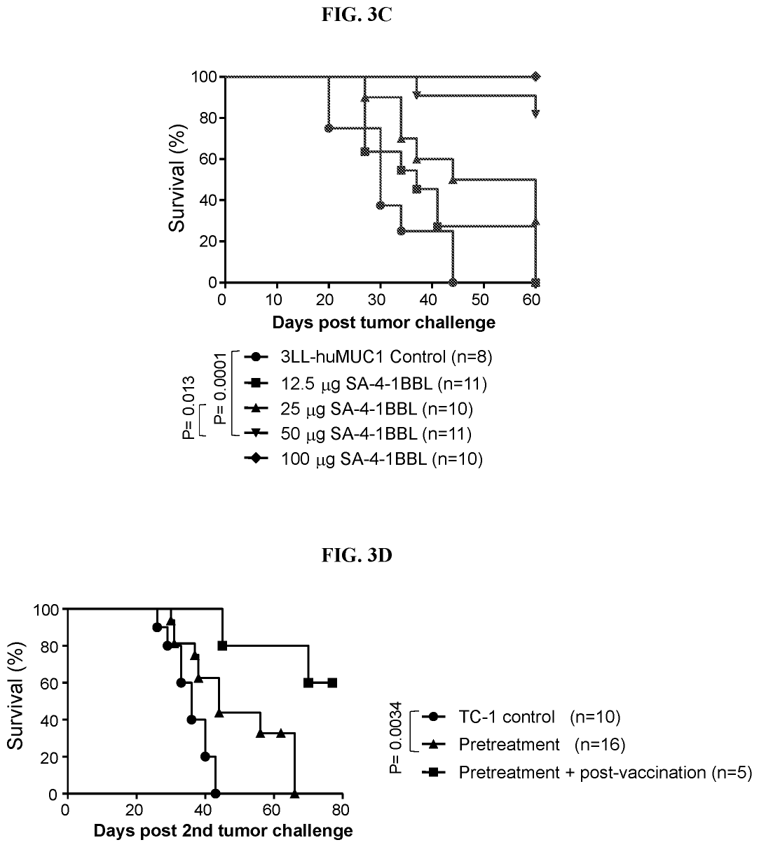

We next investigated if the protective effect of SA-4-1BBL as a single agent against TC-1 extends to other tumor types. C57BL/6 mice were pre-treated with 25 μg SA-4-1BBL twice, 2 weeks apart, followed by a challenge with LLC tumor cells 2 weeks after the second immunization (FIG. 3A). There was significant protection (p<0.0001) against LLC tumor with 40% of mice surviving over a 60-day observation period (FIG. 3B). The protection against tumor conferred by SA-4-1BBL was dose-dependent. Treatment with 100 SA-4-1BBL twice, two weeks apart followed by challenge a week later with 3LL tumor cells expressing the human mucin 1 protein (3LL-huMUC1) resulted in 100% survival over an observation period of 60 days (FIG. 3C). Treatment with smaller 12.5, 25, and 50 μg doses of SA-4-1BBL resulted in lower rates of survival 0%, 30%, and 80%, respectively. Flow cytometric analyses demonstrated the lack of expression of 4-1BB on the all 3 tumor cell lines (data not shown), ruling out the possibility of SA-4-1BBL directly targeting tumor cells for the observed protective effect.

We have previously shown that SA-4-1BBL-adjuvanted subunit vaccines generate a long-term immune memory response, primarily driven by CD8⁺ T cells, against tumors. To test whether the prophylactic efficacy of SA-4-1BBL leads to a long-lasting immune memory, mice free of TC-1 tumor for 60 days were re-challenged with a second dose of tumor cells and monitored for tumor growth. There was a significant (P=0.0034) delay in tumor growth as compared with the controls, but all mice eventually expired from tumor burden (FIG. 3D). In contrast and consistent with our previously published data, mice that had eradicated tumors in response to SA-4-1BBL-adjuvanted P1 subunit vaccine showed immune memory with 60% of animals surviving for an observation period of 80 days post second tumor challenge (FIG. 3D). These results show that the preventive immune response generated by SA-4-1BBL as a single agent lacks the characteristics of a classical adaptive immune response against tumors that benefit from a long-lasting memory.

An Agonist Antibody to 4-1BB does not Confer Protection Against Tumor Challenge

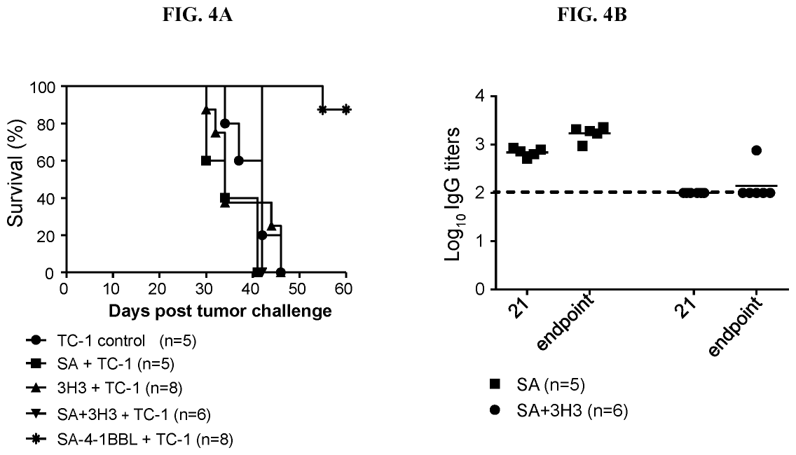

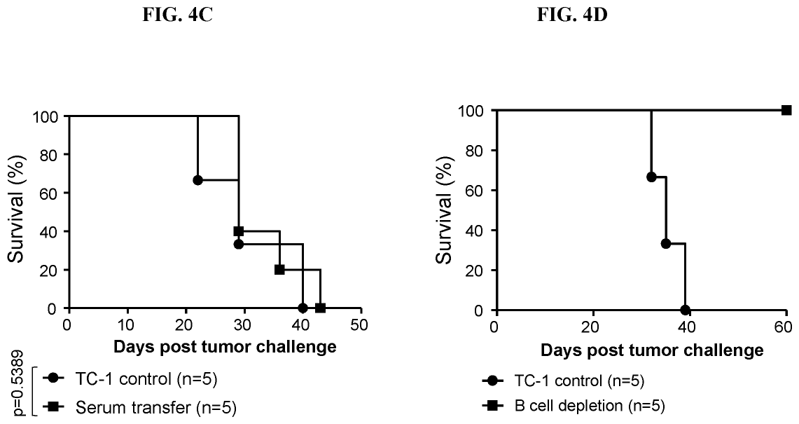

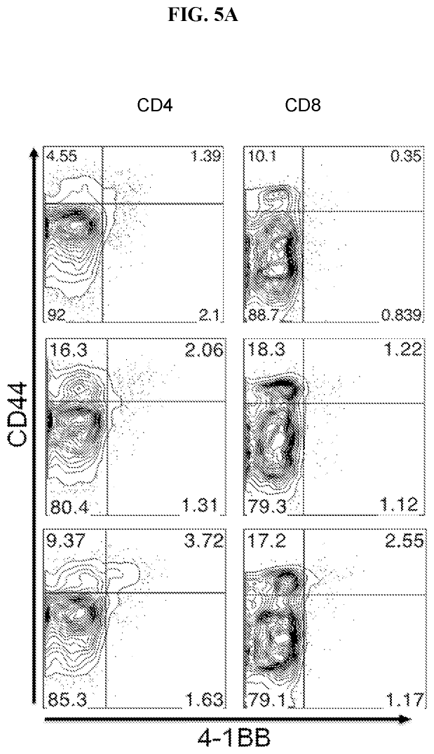

Agonistic antibodies to 4-1BB have been used for cancer immunotherapy successfully in preclinical models and are presently being tested in clinical cancer trials. Previous studies from our laboratory reported qualitative and quantitative differences between SA-41BBL and an agonistic antibody, 3H3, to 4-1BB receptor. We, therefore, assessed if the pretreatment with 3H3 antibody generates tumor preventive immune responses. C57BL/6 mice were treated twice with 3H3 (100 μg/injection, 2 weeks apart) followed by TC-1 tumor challenge two weeks after the second antibody treatment. Surprisingly, pretreatment with the agonistic antibody alone did not protect mice against tumor challenge, as all mice developed tumors in a similar tempo to untreated controls (FIG. 4A). To eliminate the potential contribution of streptavidin as a foreign antigen to the SA-4-1BBL-induced protection, a separate group of mice was pretreated with 3H3 plus an equimolar amount of streptavidin in the treatment with SA-41BBL. Streptavidin alone or in combination with 3H3 antibody did not protect the mice against TC-1 tumor challenge. In marked contrast and consistent with data presented in FIG. 2, pretreatment with SA-4-1BBL (25 μg/injection, 2 weeks apart) resulted in ˜90% survival for a 60-day observation period (FIG. 4A).

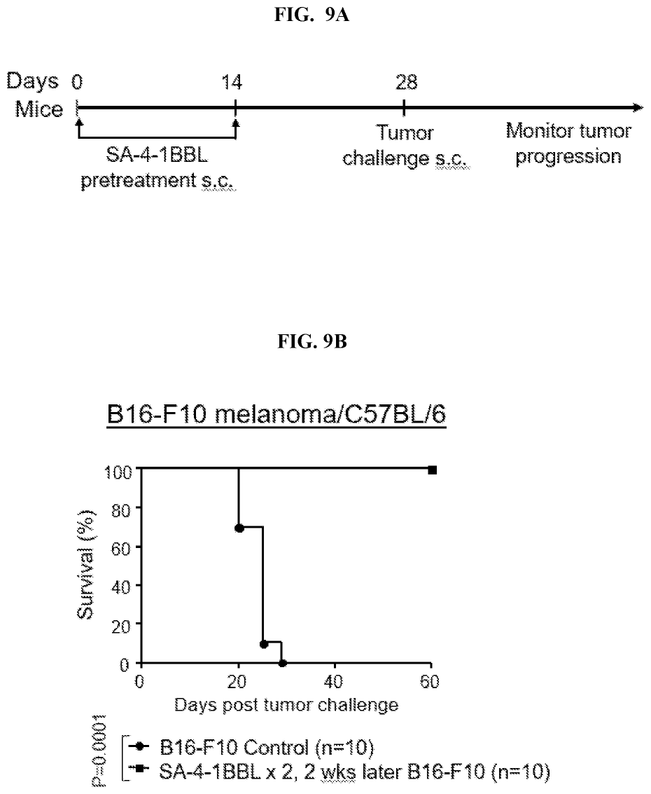

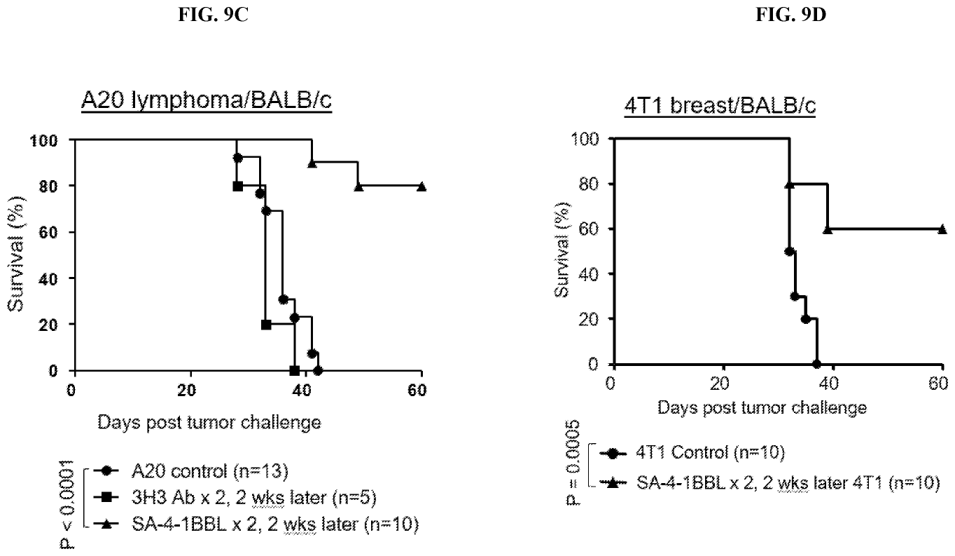

Pretreatment with SA-4-1BBL was also shown to generate protective immune responses against B16-F10 melanoma, A20 lymphoma, and triple negative breast cancer cell line challenges (FIG. 9). C57BL/6 or BALB/c mice were treated subcutaneously with SA-4-1BBL protein or the agonistic 3H3 antibody against 4-1BB receptor, twice (100 μg/injection) 2 weeks apart (FIG. 9A). Mice were challenged subcutaneously in the left back flank with live 1×10⁵ B16-F10 melanoma, 1×10⁶ A20 lymphoma, or 5×10⁴ 4T1 triple negative breast cancer cell lines as indicated. Animals were monitored for tumor growth twice a week and tumor sizes were measured using calipers. Animals were euthanized at a 60-day experimental endpoint or when tumors ulcerated or reached a size of approximately 12 mm in diameter.

Treatment with SA-4-1BBL twice, two weeks apart resulted in complete protection against B16-F10 melanoma (FIG. 9B), ˜80% protection against A20 lymphoma (FIG. 9C), and ˜60% protection against triple negative 4T1 breast cancer (FIG. 9D). Importantly, the immunoprevention was a bona fide feature of SA-4-1BBL as an agonistic AB to 4-1BB receptor (3H3) used at the same dose as SA-4-1BBL had no impact on the growth of A20 lymphoma (FIG. 9C). Taken together, these data demonstrate that SA-4-1BBL is a bona fide immune agent that train the immune system for long-lasting protection against various tumor types in two different mouse strains.

Treatment with agonistic 4-1BB antibodies was shown to block humoral immune responses by inducing anergy in CD4⁺ T cells. Consistent with these reports, none of the mice preimmunized with streptavidin and 3H3 had detectable levels of anti-streptavidin antibodies on day 21 post-treatment, and only 1/6 mice scored positive at expiration from tumor burden (FIG. 4B). In marked contrast, mice immunized twice with streptavidin protein alone had high titers of anti-streptavidin antibodies on both day 21 and experimental endpoint. Humoral immunity has been shown to play a role in the efficacy of various cancer immunotherapies. Antibodies can have a direct effect on the tumor by recognizing and binding to surface antigens, or by helping antigen-presentation and processing by APCs through opsonization, thereby augmenting downstream adaptive immune responses. Given the positive titers of anti-streptavidin antibodies in the pretreatment setting, we asked if such antibodies can contribute to the protective effect observed against tumors. Passive transfer of serum (200 μl/mouse) with high antibody titers against streptavidin into naïve C57BL/6 mice 24 hours prior to TC-1 tumor challenge did not impact tumor growth as compared with controls (FIG. 4C). To further eliminate the role of antibodies in the observed protection against tumor, mice were injected with a depleting antibody against B cells and then treated with 25 μg SA-4-1BBL twice, two weeks apart. The depletion of B cells did not negate the protective effect of SA-4-1BBL against TC-1 tumor challenge (FIG. 4D). Together, these findings reveal two substantial functional differences between agonistic antibody to 4-1BB and SA-4-1BBL. The antibody blocks humoral responses against streptavidin, and it does not protect mice against tumor challenge, whereas SA-4-1BBL shows opposite effects in both functions.

IFN-γ⁺ Producing CD4⁺ T and NK Cells as Predictors of SA-4-1BBL-Mediated Immune Protection Against Tumors

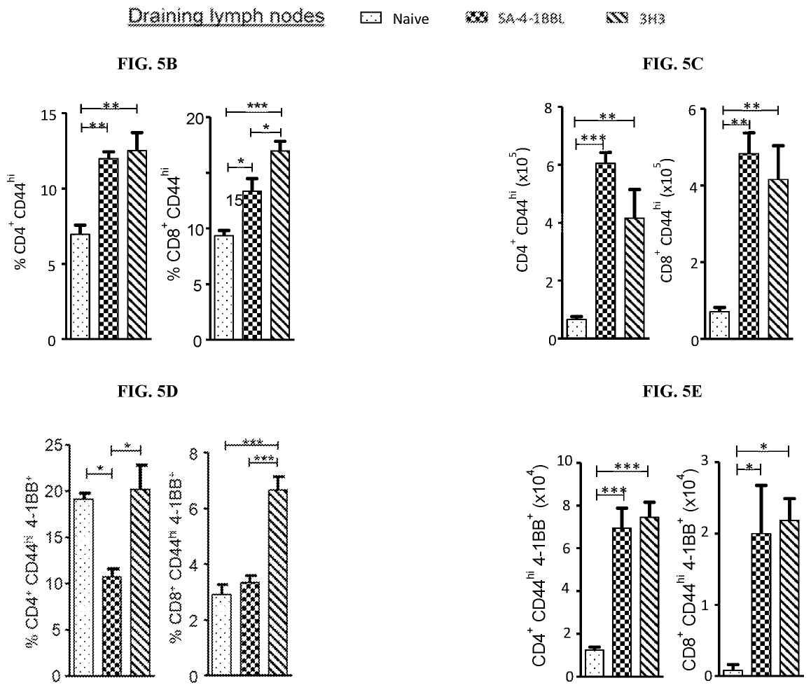

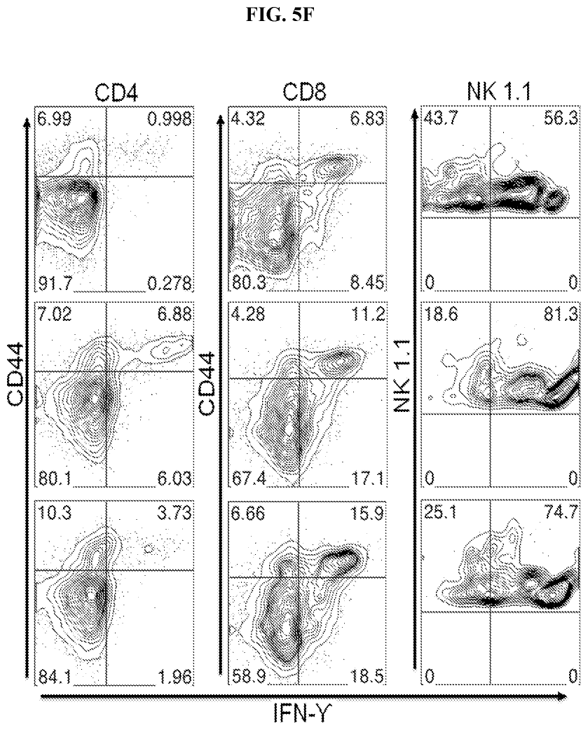

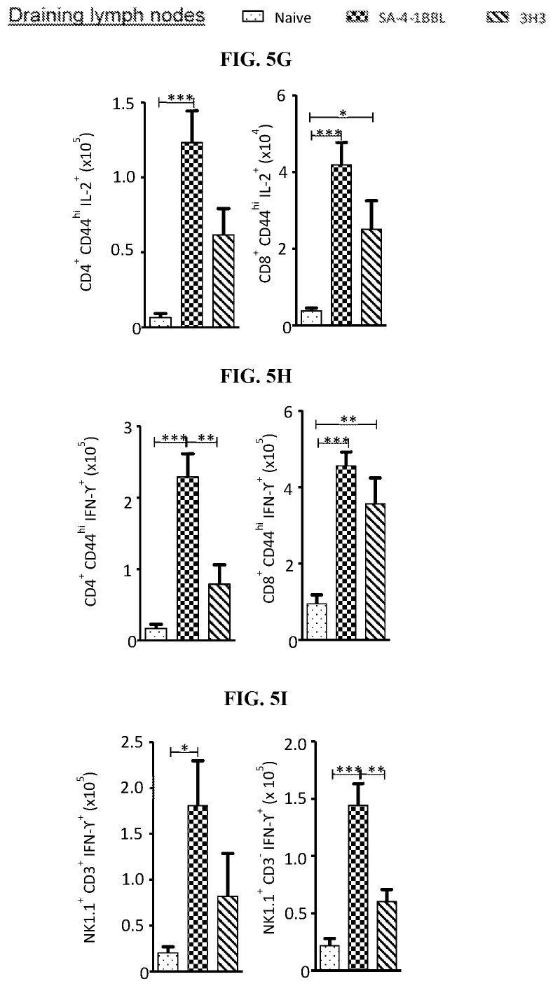

To establish immune correlates of protection against tumors and elucidate potential mechanistic differences between SA-4-1BBL and the agonistic 4-1BB antibody, mice were treated twice, two weeks apart with SA-4-1BBL or 3H3 antibody and euthanized 3 days later to collect lymphoid tissues for analyses. Naïve mice had significant percentages of CD4⁺ (˜7%) and CD8⁺ (˜10%) T cells expressing CD44 molecule as a memory marker (FIG. 5A, 5B). Treatment with SA-4-1BBL or 3H3 significantly increased the percentage and absolute numbers of both CD4⁺ and CD8⁺ T cells with memory phenotype in draining LNs as compared with naïve mice (FIG. 5B, 5C). A similar trend, particularly for the 3H3 antibody, was also observed for T cells in the spleen (data not shown). Considerable percentage of memory-like CD4⁺ (>18%) and CD8⁺ (>3%) T cells in naïve mice also expressed 4-1BB on their surface (FIG. 5A, 5D). Treatment with SA-4-1BBL or 3H3 antibody significantly increased the absolute numbers of lymph node CD4⁺ and CD8⁺ T memory cells expressing 4-1BB on their surface as compared with naïve mice (FIG. 5E). Interestingly, treatment with SA-4-1BBL decreased the percentage of memory CD4⁺ T cells expressing 4-1BB as compared with naïve mice, plausibly due to cell surface modulation of the receptor on actively expanding cells as previously reported.

Treatment with SA-4-1BBL resulted in a significant increase in the percentage and absolute number of lymph node CD4⁺CD44⁺ T cells expressing IL-2 as compared with naïve mice (FIG. 5F, 5G). In marked contrast, treatment with 3H3 antibody resulted in an increase of lymph node CD4⁺CD44⁺ T cells expressing IL-2, but it was not significant as compared with naïve mice (FIG. 5G). This trend also applied to CD8⁺CD44⁺ T cells expressing IL-2, except the difference between 3H3 and naïve was significant (FIG. 5G). SA-4-1BBL treatment also resulted in a significant increase in the percentage and absolute number of lymph node CD4⁺CD44⁺ T cells expressing IFN-γ as compared with naive and 3H3 antibody treated mice (FIG. 5H). There was a similar trend for CD8⁺CD44⁺ T cells expressing IFN-γ, but the difference between SA-4-1BBL and 3H3 antibody was not significant (FIG. 5H). Importantly, treatment with SA-4-1BBL resulted in a significant increase in the percentage and absolute numbers of NK1.1⁺CD3⁻ NK cells expressing IFN-γ as compared with naïve and 3H3 antibody treated mice (FIG. 5I). A similar trend was also observed for splenic T and NK cells, but the differences between SA-4-1BBL and 3H3 was not as pronounced as for the LN cells (data not shown). The observed differences induced by SA-4-1BBL in the number and percentages of CD4⁺CD44⁺ T cells between draining lymph nodes and spleen was not due to the differential expression of 4-1BB on these cells as determined by flow cytometry (data not shown). Together, these findings demonstrate that the SA-4-1BBL preferentially expands memory like CD4⁺CD44⁺ T cells and CD3⁻ NK cells producing IFN-γ; features that are not shared by the agonist anti-4-1BB antibody.

### SA-4-1BBL-Mediated Protection Against Tumor Appears to Require IFN-γ as a Mediator of Cross-Talk Between NK and CD4⁺ T Cells

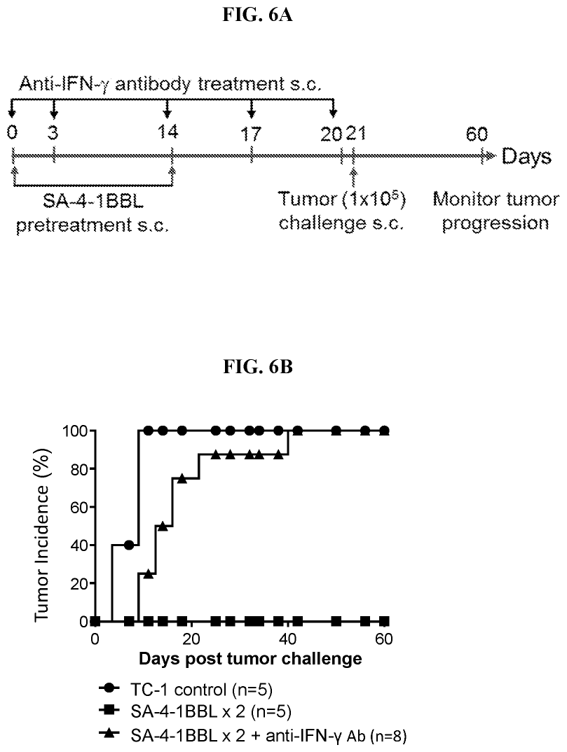

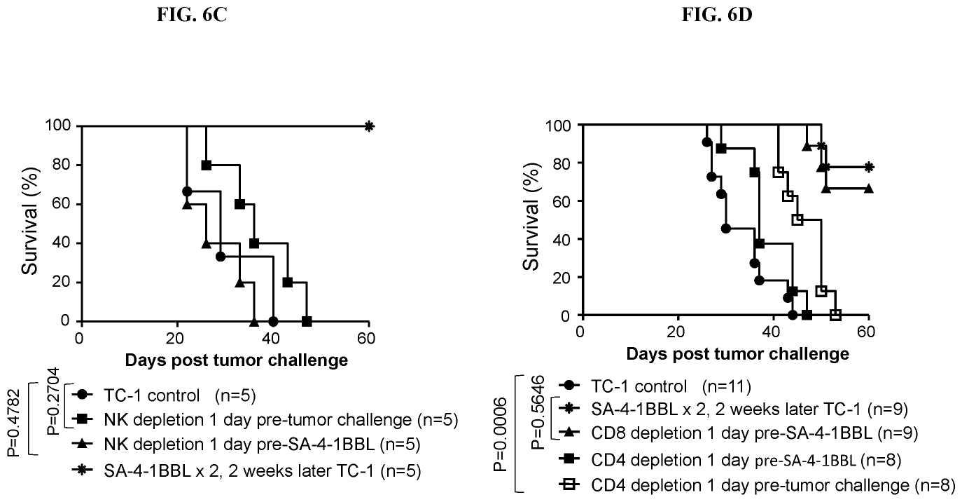

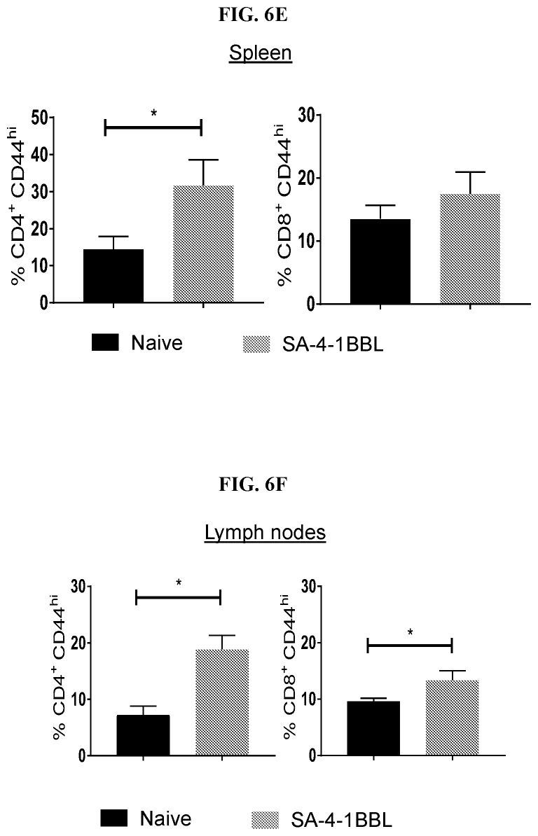

The significant increase in the frequency of IFN-γ production in response to SA-4-1BBL treatment as compared with 3H3 antibody led us to investigate the involvement of this cytokine in the observed tumor prevention. Mice were pretreated s.c. with SA-4-1BBL (100 μg/injection) twice, two weeks apart, followed by TC-1 s.c. challenge (1×10⁵ cells/animal) one week later. Animals were also treated with a blocking antibody to IFN-γ (200 μg/injection) for a total of 5 doses on days 0, 3, 14, 17, 20 in reference to SA-4-1BBL treatment (FIG. 6A). All mice treated with anti-FN-γ antibody developed tumor in a delayed fashion (day 42) as compared with control mice (day 9; FIG. 6B). Importantly, none of the mice pretreated with SA-4-1BBL developed tumor in a 60-day observation period.

The significant increase in the absolute number of NK cells and CD4⁺ T cells expressing IFN-γ in SA-4-1BBL treated mice as compared with agonistic 3H3 antibody led us to directly probe the contribution of these cell populations to protection against tumor. Depletion of NK cells using an antibody to NK1.1 molecule one day before treatment with SA-4-1BBL (priming phase) overcame protection against TC-1 tumor as both SA-4-1BBL-treated and untreated control mice showed a similar tumor growth tempo (FIG. 6C). NK cell depletion one day before tumor challenge (day 27, effector phase) also resulted in complete ablation of the protective effect of SA-4-1BBL, providing direct evidence for the role of NK as effector cells.

Depletion of CD4⁺ T cells one day prior to SA-4-1BBL treatment also resulted in the abrogation of anti-tumor protective effect in the TC-1 model (FIG. 6D). Although the depletion of this cell population 1 day before TC-1 challenge resulted in tumor growth in all mice, there was a significant (P=0.0006) delay in tumor progression as compared with controls. In marked contrast, the depletion of CD8⁺ T cells had no impact on the anti-tumor effect of SA-4-1BBL (FIG. 6D). Immune monitoring of T cells in mice that cleared their tumor in the SA-4-1BBL pretreatment group demonstrated significantly increased frequency of memory CD4⁺ T cells in the spleen (FIG. 6E) and in the draining lymph nodes (FIG. 6F) analyzed 60 days post-tumor challenge as compared with naïve mice. The frequency of CD8⁺ T cells remained unchanged in the spleen but was significantly increased in the draining lymph nodes as compared with naive animals (FIG. 6E, F). These data demonstrate the obligatory effect of NK and CD4⁺ T cells as well as IFN-γ to sustain SA-4-1BBL-generated protection against tumors.

### SA-4-1BBL Prevents Post-Surgical Tumor Recurrences

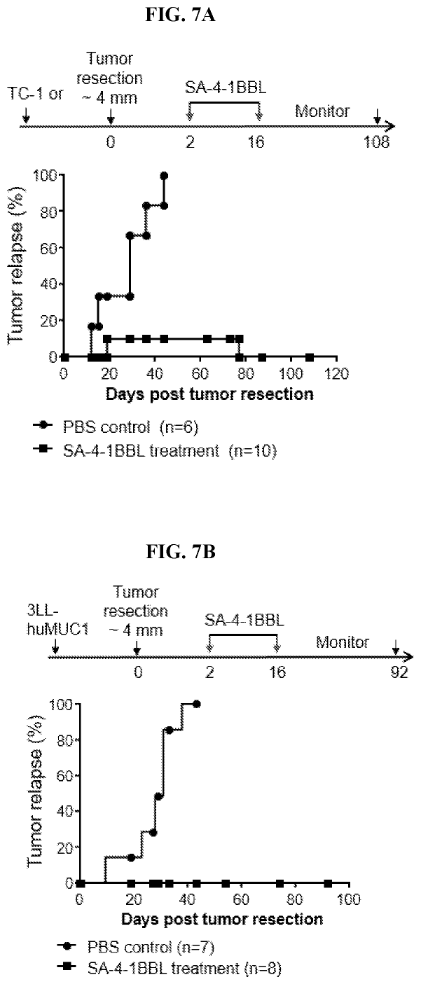

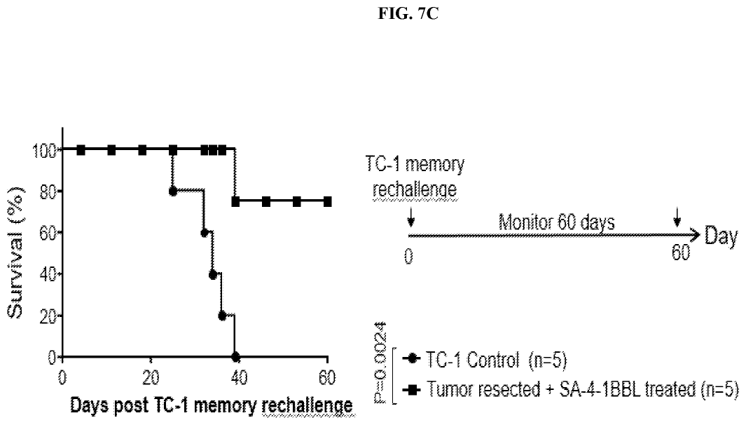

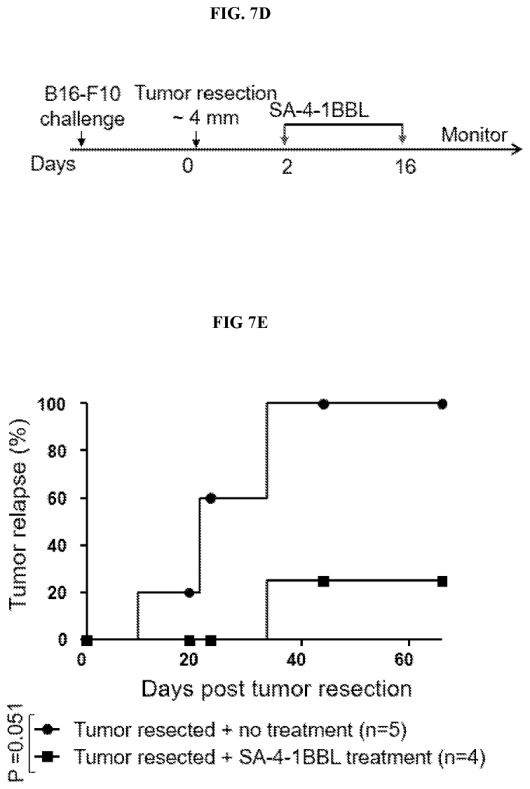

Post-surgical tumor recurrence is a significant hurdle in cancer treatment. The observed preventive effect of SA-4-1BBL across various tumor types led us to test the efficacy of this molecule in controlling tumor recurrence in clinically relevant surgical resection models. C57BL/6 mice with established TC-1 tumors (˜4 mm in diameter) were subjected to surgery to debulk the tumor. Animals were randomly assigned to treatment and control groups. Treatment with SA-4-1BBL (25 μg/injection, 2 weeks apart) resulted in complete blockade of tumor recurrence, while all controls had tumor relapse and expired from tumor burden within 50 days post-tumor resection (FIG. 7A). Importantly, one out of ten mice in the SA-4-1BBL treatment group had relapsed tumor, which was eventually eradicated (FIG. 7A). Treatment with the same SA-4-1BBL regimen also controlled post-surgical recurrences in the 3LL-huMUC1 tumor model (FIG. 7B). Importantly, 80% of mice that cleared primary tumors in response to post-surgical treatment with SA-4-1BBL did not develop tumor when re-challenged with TC-1 cells, demonstrating the establishment of long-term immune memory (FIG. 7C).

The effect of SA-4-1BBL on preventing post-surgical tumor recurrence was also tested in clinically relevant C57BL/6 mice with established B16-F10 melanoma tumors. C57BL/6 mice with established B16-F10 tumors (approximately 4 mm in diameter) were subjected to surgery to debulk the tumor under sterile conditions. After 48 hours of recovery period, animals were treated subcutaneously with SA-4-1BBL protein (100 μg/injection) twice, 2 weeks apart. Animals without any treatment served as controls and mice were monitored for tumor relapse. Treatment with SA-4-1BBL resulted in blockade of tumor recurrence in 75% of mice, while all controls had tumor relapse and expired from tumor burden within 70 days post-tumor resection (FIG. 7D-7E). These results demonstrate that SA-4-1BBL as monotherapy is effective in controlling post-surgical tumor recurrences by generating effective adaptive immune responses.

Collectively, these results demonstrate that SA-4-1BBL as monotherapy is effective in controlling post-surgical tumor recurrences by generating effective and long-lasting adaptive immune responses.

What is claimed is:

1. A monotherapy method of preventing or treating a cancer, comprising administering to a subject in need thereof an effective amount of SA-4-1BBL, wherein the monotherapy method does not include administering to the subject an antigen associated with the cancer.

2. A monotherapy method of reducing the risk of tumorigeneses, comprising administering to a subject in need thereof, an effective amount of SA-4-1BBL, wherein the monotherapy method does not include administering to the subject an antigen associated with the tumor.

3. A monotherapy method of reducing the risk of tumor recurrence, comprising administering to a subject in need thereof, an effective amount of SA-4-1BBL, wherein the monotherapy method does not include administering to the subject an antigen associated with the tumor.

4. A method according to claim 4, wherein the subject has undergone surgical removal of tumor cells, chemotherapy, and/or cancer irradiation treatment.

5. A method according to any one of claims 1-4, wherein the subject is suffering from chronic liver disease, has a hereditary mutation in a p53 gene, has a hereditary mutation in a breast cancer gene, has a DNA repair deficiency, and/or has preneoplastic or early neoplastic lesions.

6. A method according to any one of claims 1-4, wherein the subject has lifestyle risk factors, environmental risk factors, a history of hereditary BRCA1 and/or BRCA2 mutations, Lynch Syndrome, Cowden Syndrome, and/or is infected with HPV.

7. A method according to any one of claims 1-4, wherein the SA-4-1-BBL is administered by intravenous, subcutaneous, or intraperitoneal injection.

8. A method according to any one of claims 1-4, wherein the SA-4-1-BBL is administered twice, two weeks apart.

9. The method of claim 8, wherein the method further comprises a rest period of 2-6 months, after which the SA-4-1-BBL is administered twice, two weeks apart.

10. A method according to any one of claims 1-4, wherein the SA-4-1-BBL comprises the amino acid sequence of the extracellular domain of human 4-1-BBL.

11. A method according to any one of claims 1-4, wherein the SA-4-1-BBL comprises the amino acid sequence set forth in FIG. 8B.

12. An SA-4-1-BBL conjugate having the amino acid sequence set forth in FIG. 8A or FIG. 8B.

13. A composition comprising a SA-4-1-BBL conjugate according to claim 12 and a carrier.

14. A pharmaceutical composition comprising an SA-4-1-BBL conjugate according to claim 12 and a pharmaceutically acceptable carrier.

15. A pharmaceutical composition according to claim 14, wherein the pharmaceutically acceptable carrier is suitable for administration by intravenous, subcutaneous, or intraperitoneal injection.

16. A SA-4-1BBL conjugate for a monotherapy method for preventing or treating a cancer, reducing the risk of tumorigeneses, and/or reducing the risk of tumor recurrence, wherein the conjugate has the amino acid sequence set forth in FIG. 8A or FIG. 8B, and the monotherapy method does not include administering to the subject an antigen associated with the cancer or tumor.
